(19) United States
(12) Patent Application Publication
(10) Pub. No.: US 2016/0334636 A1
(43) Pub. Date: Nov. 17, 2016

(54) LIGHT FIELD FILTER

(71) Applicants: Zhen Xiao, Beijing (CN); EMPIRE TECHNOLOGY DEVELOPMENT LLC, Wilmington, DE (US)

(72) Inventor: Zhen Xiao, Beijing (CN)

(73) Assignee: EMPIRE TECHNOLOGY DEVELOPMENT LLC, Wilmington, DE (US)

(21) Appl. No.: 15/110,953

(22) PCT Filed: Jan. 27, 2014
(86) PCT No.: PCT/CN2014/071542

§ 371 Date: Jul. 12, 2016

(51) Int. Cl. G02B 27/46; G02B 25/00; A61F 9/00; G02C 7/04; G02C 7/02

(52) U.S. Cl. CPC G02B 27/46; G02C 7/043; G02C 7/022; A61F 9/0017; G02B 25/001; G02C 2202/20

(57) ABSTRACT

According to some examples, a lens apparatus includes a plurality of near-field spatial filters, each of the near-field spatial filters including a first near-field lens that includes a first focal length, a second near-field lens that includes a second focal length, and a first plate that includes a pin-hole, the first plate being disposed between the first near-field lens and the second near-field lens such that the pin-hole in the first plate is substantially at a point where a focus of the first near-field lens and a focus of the second near-field lens substantially coincide based at least in part on the first focal length and the second focal length. The lens apparatus further includes a plurality of far-field spatial filters optically coupled to the plurality of near-field spatial filters, each of the far-field spatial filters including a first field lens that includes a third focal length, a second far-field lens that includes a fourth focal length, and a second plate that includes a pin-hole, the second plate being disposed between the first far-field lens and the second far-field lens such that the pin-hole in the second pin-hole plate is substantially at a point where a focus of the first far-field lens and a focus of the second far-field lens substantially coincide based at least in part on the third focal length and the fourth focal length, and wherein the third focal length and the fourth focal length are different than either of the first focal length and the second focal length.

### BACKGROUND

Unless otherwise indicated herein, the materials described in this section are not prior art to the claims in this application and are not admitted to be prior art by inclusion in this section.

In current systems, resolving images of objects at close range and at a distance in a single image may be difficult. The depth of field can be increased by use of some methods. However, such methods may not resolve images of all objects in an image if the distance between the far-field object and the near-field object is very long. For example, the method of light field camera imaging focuses an image after capturing and selectively resolves objects within the image having different distances. However, light field camera imaging may not be executed in real-time.

Further, when an observer tries to focus his/her eyes on a scene having far-field and near-field objects, ciliary muscles may become fatigued thereby causing asthenopia and/or other eye discomfort. Such problems may be significant when using head-up display (HUD) and/or microscopes.

### SUMMARY

In an example, a lens apparatus may comprise, a plurality of near-field spatial filters, each of the near-field spatial filters including a first near-field lens that includes a first focal length, a second near-field lens that includes a second focal length, and a first plate that includes a pin-hole, the first plate being disposed between the first near-field lens and the second near-field lens such that the pin-hole in the first plate is located substantially at a point where a focus of the first near-field lens and a focus of the second near-field lens substantially coincide based at least in part on the first focal length and the second focal length and a plurality of far-field spatial filters optically coupled to the plurality of near-field spatial filters, each of the far-field spatial filters including a first far-field lens that includes a third focal length, a second far-field lens that includes a fourth focal length, and a second plate that includes a pin-hole, the second plate being disposed between the first far-field lens and the second far-field lens such that the pin-hole in the second pin-hole plate is located substantially at a point where a focus of the first far-field lens and a focus of the second far-field lens substantially coincide based at least in part on the third focal length and the fourth focal length, and wherein the third focal length and the fourth focal length are different than either of the first focal length and the second focal length.

In an example, an apparatus may comprise, a first spatial filter, the first spatial filter including a first lens that includes a first focal length, a second lens that includes a second focal length, and a first plate that includes a pin-hole, the first plate being disposed between the first lens and the second lens such that the pin-hole in the first plate is located substantially at a point where a focus of the first lens and a focus of the second lens substantially coincide based at least in part on the first focal length and the second focal length, and a second spatial filter optically coupled to the first spatial filter, the second spatial filter including a third lens that includes a third focal length, a fourth lens that includes a fourth focal length, and a second plate that includes a pin-hole, the second plate being disposed between the third lens and the fourth lens such that the pin-hole in the second plate is located substantially at a point where a focus of the third lens and a focus of the fourth lens substantially coincide based at least in part on third focal length and the fourth focal length.

In an example, a system may comprise a first spatial filter, the first spatial filter including a first lens that includes a first focal length, a second lens that includes a second focal length, and a first optical processing filter, the first optical processing filter being disposed between the first lens and the second lens such that the optical processing filter is located substantially at a point where a focus of the first lens and the focus of the second lens substantially coincide based at least in part on the first focal length and the second focal length. The system may further comprise a second spatial filter optically coupled to the first spatial filter, the second spatial filter including a third lens that includes a third focal length, a fourth lens that includes a fourth focal length, and a second optical processing filter, the second optical processing filter being disposed between the third lens and the fourth lens such that the second optical processing filter is located substantially at a point where a focus of the third lens and the focus of the fourth lens substantially coincide based at least in part on the third focal length and the fourth focal length. The system may also comprise an optical processing filter control module operatively coupled to the first and second optical processing filters, the optical processing control module operatively enabled to block a zero-order diffraction of light incident at the first optical processing filter and the second optical processing filter and enable first-order diffraction of light incident at the first optical processing filter and the second optical processing filter.

In an example, a method is provided to filter light by use of a first spatial filter, the first spatial filter including a first lens that includes a first focal length, a second lens that includes a second focal length, and a first optical processing filter, the first optical processing filter being disposed between the first lens and the second lens such that the first optical processing filter is located substantially at a point where a focus of the first lens and a focus of the second lens substantially coincide based at least in part on the first focal length and the second focal length; and by use of a second spatial filter optically coupled to the first spatial filter, the second spatial filter including a third lens that includes a third focal length, a fourth lens that includes a fourth focal length, and a second optical processing filter, the second optical processing filter being disposed between the third lens and the fourth lens such that the second optical processing filter is located substantially at a point where a focus of the third lens and a focus of the fourth lens substantially coincide based at least in part on third focal length and the fourth focal length. The method may comprise blocking a zero-order diffraction of light incident at the first optical processing filter and the second optical processing filter and enabling a first-order diffraction of light incident at the first optical processing filter and the second optical processing filter.

In an example, a process to manufacture a micro-lens array may comprise forming a pair of first lenses that include a first focal length, forming a pair of second lenses that include a second focal length, forming first and second plates that include a pin-hole in each plate, affixing the first plate between the pair of first lenses such that the pin-hole in the first plate is substantially at a point where a focus of the pair of first lenses substantially coincide, affixing the second plate between the pair of second lenses such that the pin-hole in the second plate is substantially at a point where a focus of the pair of second lenses substantially coincide and forming a viewer apparatus from the pair of first lenses including the first plate and the pair of second lenses including the second plate, wherein the viewer apparatus is configured to optically couple the pair of first lenses including first plate and the pair of second lenses including the second plate.

In an example, a non-transitory machine-readable medium includes instructions stored thereon, which in response to execution by one or more processors, may enable an optical processing filter module to perform or cause to be performed the method of any of the example processes or methods described above and/or herein.

The foregoing summary is illustrative only and not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure, and are therefore, not to be considered limiting of its scope. The disclosure will be described with additional specificity and detail through use of the accompanying drawings.

In the drawings:

FIG. 1A illustrates an example of a lens to filter light reflected from (or otherwise returned/directed from) near-field and far-field objects;

FIG. 1B illustrates an example of a lens to filter light reflected from (or otherwise returned/directed from) near-field and far-field objects;

FIG. 2A is a diagram of example focal distances of a filter to filter light reflected from (or otherwise returned/directed from) near-field and far-field objects in an example lens;

FIG. 2B is a diagram of example focal distances of a filter to filter light reflected from (or otherwise returned/directed from) near-field and far-field objects in an example lens;

FIG. 3 illustrates a flowchart of an example process to filter light reflected from (or otherwise returned/directed from) near-field and far-field objects in an example lens;

FIG. 4 illustrates a flowchart of an example process to make a lens to filter light reflected from (or otherwise returned/directed from) near-field and far-field objects in an example lens;

FIG. 5 illustrates an example computer program product to control or otherwise operate in conjunction with an example lens to filter light reflected from (or otherwise returned/directed from) near-field and far-field objects; and

FIG. 6 illustrates a block diagram of an example computing device, all arranged in accordance with at least some embodiments described herein.

### DETAILED DESCRIPTION

The following description sets forth various examples along with specific details to provide a thorough understanding of the subject matter. The subject matter may be practiced without some or more of the specific details disclosed herein. Further, in some circumstances, well-known methods, procedures, systems, components and/or circuits have not been described in detail, in order to avoid unnecessarily obscuring the subject matter.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. The aspects of the present disclosure, as generally described herein, and illustrated in the Figures, can be arranged, substituted, combined, and designed in a wide variety of different configurations, all of which are explicitly contemplated and made part of this disclosure.

This disclosure is drawn, inter alia, to technologies including methods, devices, systems and computer readable media related to filtering light reflected from (or otherwise returned/directed from) near-field and far-field objects in one or more lenses.

Briefly stated, according to some examples, a lens apparatus includes a plurality of near-field spatial filters, each of the near-field spatial filters including a first near-field lens that includes a first focal length, a second near-field lens that includes a second focal length, and a first plate that includes a pin-hole, the first plate being disposed between the first near-field lens and the second near-field lens such that the pin-hole in the first plate is substantially at a point where a focus of the first near-field lens and a focus of the second near-field lens substantially coincide based at least in part on the first focal length and the second focal length. The lens apparatus further includes a plurality of far-field spatial filters optically coupled to the plurality of near-field spatial filters, each of the far-field spatial filters including a first field lens that includes a third focal length, a second far-field lens that includes a fourth focal length, and a second plate that includes a pin-hole, the second plate being disposed between the first far-field lens and the second far-field lens such that the pin-hole in the second pin-hole plate is substantially at a point where a focus of the first far-field lens and a focus of the second far-field lens substantially coincide based at least in part on the third focal length and the fourth focal length, and wherein the third focal length and the fourth focal length are different than either of the first focal length and the second focal length.

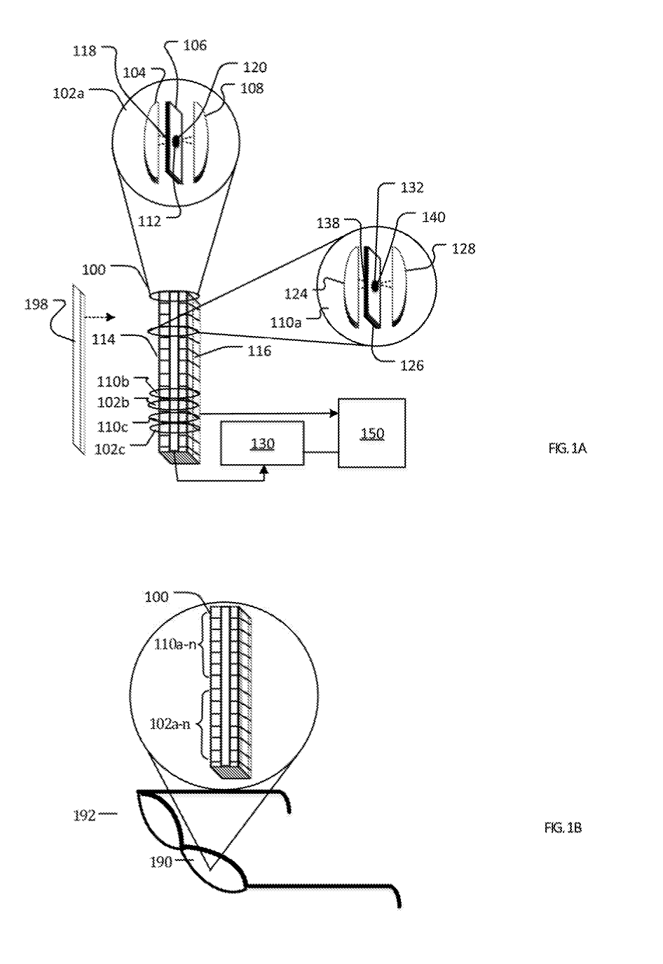

FIG. 1A illustrates an example of a lens to filter light reflected from (or otherwise returned/directed from) near-field and far-field objects. More specifically, FIG. 1A illustrates an example of a lens 100 configured to filter light reflected from (or otherwise returned/directed from) near-field and far-field objects. Lens 100 may be an apparatus that comprises a near-field spatial filter 102a and a far-field spatial filter 110. In an example, near-field spatial filter 102a may be configured to focus on objects that are close to lens 100 and far-field spatial filter 110a may be configured to focus on objects that are distant from lens 100. Distant objects may be farther from lens 100 than close objects. In an example, close may be a distance within about six meters of lens 100 and distant may be a distance greater than about six meters from lens 100. However, in another example, the term “close” may be interpreted to mean farther away or closer than six meters and the term “distant” may be interpreted to mean farther away or closer than six meters. As an example, being within or further away than approximately five, seven, eight etc. meters may be the nominal distance from which to gauge an object as being “close” or “distant,” rather than using six meters as the nominal distance.

In an example, near-field spatial filter 102a may comprise a first near-field lens 104, a second near-field lens 108 and a first light filter 106. First near-field lens 104 may comprise a lens (e.g., a microlens) in a first microlens array 114. Second near-field lens 108 may comprise a lens (e.g., a microlens) in a second microlens array 116.

In an example, first near-field lens 104 may be configured for near-field focus at a first focal length. Second near-field lens 108 may be configured for near-field focus at a second focal length. In an example, the first focal length and the second focal length may be the same or different.

In an example, first light filter 106 may be disposed between first near-field lens 104 and second near-field lens 108. First light filter 106 may comprise a plate including a pinhole 112 (or other opening) configured to block light that is not focused within an aperture of pinhole 112. The term “divergent light” may refer to light that is divergent to an entrance of lens 100. For example, light reflecting from (or otherwise returned/directed from) a distant object may be divergent light. When such divergent light is passing through first near-field lens 104, the divergent light may be refracted through (or otherwise pass through) the first near-field lens 104 and may be focused behind pinhole 112. In such a case, a substantial portion of the light may be blocked by the pinhole plate provided by first light filter 106. The power of divergent light that travels through the second near-field lens 108 may be scattered by second near-field lens 108. Thus, most of the power of the light from a close-range object may not enter into an optical image system 150. The divergent light may be refracted away from pinhole 112 by first near-field lens 104 and thus may be blocked by first light filter 106.

In one example, first light filter 106 may not include pinhole 112 and rather may comprise an optical processing filter configured to process optical information and may operate in real-time to perform image processing. Various optical processing filters may be suitable. For example, a high-pass filter (e.g., to provide edge enhancement), a polarization plate, a color filter, and/or a direction selection filter and/or other filters may be used. In an example, where first light filter 106 comprises an optical processing filter, an optical processing filter control module 130 may be coupled and/or otherwise in communication with first light filter 106. Optical processing filter control module 130 may be configured to block zero-order diffraction of light incident at first light filter 106 and allow (e.g., not block) first-order diffraction of light incident at first light filter 106. In an example, optical processing filter control module 130 may be configured to communicate with optical image system 150, for example, to send and/or receive/detect commands and/or optical processing data.

In an example, first light filter 106 may be positioned based at least in part on the first focal length and the second focal length. In an example, a near-field back focus 118 of first near-field lens 104 may be positioned at the first focal length from first near-field lens 104. In an example, near-field back focus 118 may be the back focus point of first near-field lens 104. A near-field front focus 120 may be positioned at the second focal length from second near-field lens 108. In an example, near-field front focus 120 may be the front focus point of second near-field lens 108. Near-field back focus 118 may substantially coincide with near-field front focus 120. Pinhole 112 may be positioned about where near-field back focus 118 and near-field front focus 120 substantially coincide to form near-field spatial filter 102a.

In an example, far-field spatial filter 110a may comprise a first far-field lens 124, a second far-field lens 128 and a second light filter 126. First far-field lens 124 may comprise a lens (e.g., a microlens) in first microlens array 114. Second far-field lens 128 may comprise a lens (e.g., a microlens) in second microlens array 116. First far-field lens 124 may be positioned adjacent to first near-field lens 104 in first microlens array 114. Second far-field lens 128 may be positioned adjacent to second near-field lens 108 in second microlens array 116. First near-field lens 104, first far-field lens 124, second near-field lens 108 and second far-field lens 118 may be disposed at other locations in first microlens array 114 and second microlens array 116. For example, first near-field lens 104 and first far-field lens 124 may be positioned in a repeating or random pattern in first microlens array 114 and second near-field lens 108 and second far-field lens 118 may be positioned in a repeating or random pattern in second microlens array 116.

In an example, first far-field lens 124 may be configured for far-field focus at a third focal length. Second far-field lens 128 may be configured for far-field focus at a fourth focal length. In an example, the third focal length and the fourth focal length may be the same or different.

In an example, second light filter 126 may be disposed between first far-field lens 124 and second far-field lens 128. Second light filter 126 may comprise a plate including a pinhole 132 (or other opening) configured to block light that is not focused within an aperture of pinhole 132. For example, light reflecting from (or otherwise returned/directed from) a close-range object and passing through first far-field lens 124 may be refracted away from pinhole 132 by first far-field lens 124 and thus may be blocked by second light filter 126.

In one example, second light filter 126 may not include pinhole 132 and rather may comprise an optical processing filter configured to process optical information in substantially real-time. In an example, where second light filter 126 comprises an optical processing filter, the optical processing filter control module 130 may be coupled and/or otherwise in communication with second light filter 126. Optical processing filter control module 130 may be configured to block zero-order diffraction of light incident at second light filter 126 and allow (e.g., not block) first-order diffraction of light incident at second light filter 126.

In an example, a plate comprising pinhole 132 and/or a plate comprising pinhole 112 may be replaced by one or more other Fourier optical processing filters, thereby more optical information processing effects may be achieved, for example the phase difference imaging by blocking the zero-order diffraction and allowing the first-order diffraction to pass through.

In an example, second light filter 126 may be positioned based at least in part on the third focal length and the fourth focal length. A far-field back focus 138 of first far-field lens 124 may be positioned at the third focal length from first far-field lens 124. Far-field back focus 138 may be the back focus point of first far-field lens 124. A far-field front focus 140 may be positioned at the fourth focal length from second far-field lens 128. In an example, far-field front focus 140 may be the front focus point of second far-field lens 128. Far-field back focus 138 may substantially coincide with far-field front focus 140. Pinhole 132 may be positioned about where far-field back focus 138 and far-field front focus 140 substantially coincide to form far-field spatial filter 110a. In an example, near-field spatial filter 102a and far-field spatial filter 110a may form a 4f spatial filter.

In an example, lens 100 may include a plurality of near-field spatial filters 102a-c and far-field spatial filters 110a-c. One or more of near-field spatial filters 102a-c and far-field spatial filters 110a-c may be arranged in a variety of optical devices/systems. For example, at least near-field spatial filter 102a and far-field spatial filter 110a, may be incorporated into a viewer/viewing apparatus of a contact lens, an intraocular lens, an eyeglass, a windshield, goggles, a telescope, a microscope (e.g., a phase contrast microscope), a camera, and/or a car/building window and other viewing/viewer apparatus or a combination thereof.

In an example, first near-field lens 104, second near-field lens 108, first far-field lens 124 and second far-field lens 128 may comprise a variety of materials, such as, for example, glass, plastic, crystal, liquid crystal, and/or materials with variable refractive indexes, and/or other materials or combinations thereof. Examples of such materials with variable refractive indexes may include liquid crystal in which the refractive index can be changed by voltage and/or a solution in which the refractive index may be changed by the salinity. Such materials with variable refractive indexes may be bored and/or filled between thin layers of solid material in order to produce any of first near-field lens 104, second near-field lens 108, first far-field lens 124 and second far-field lens 128. Thus, refractive indexes of first near-field lens 104, second near-field lens 108, first far-field lens 124 and second far-field lens 128 may be made adjustable. First near-field lens 104, second near-field lens 108, first far-field lens 124 and/or second far-field lens 128 may comprise a variety of lenses, for example, a gradient-index (GRIN) lens, a Fresnel lens and/or a Fresnel zone plate or various other suitable lenses, materials and/or arrays or combinations thereof.

In another example, an image inversion lens 198 may be disposed before, after, or in lens 100 or may be disposed in another location. Image inversion lens 198 may be configured to invert images viewed through either or both of the microlens arrays 114 and 116 of lens 100.

Near-field spatial filters 102a-c and far-field spatial filters 110a-c may be arranged with respect to one another in any of a variety of ways in lens 100. For example, first spatial filters 102a-c and second spatial filter 110a-c may be arranged randomly or in a pattern. The pattern may alternate, or be arranged in other ways. Near-field spatial filters 102*-c and far-field spatial filters 110a-c may be arranged according to how light may be reflected from (or otherwise returned/directed from) a near-field and/or far-field object. In one example, there may be equal numbers of near-field spatial filters and far-field spatial filters in lens 100. In another example, there may be unequal numbers of near-field spatial filters and far-field spatial filters in lens 100. Near-field spatial filters 102a-c and far-field spatial filters 110a-c may be arranged according to an intended use of a viewing apparatus incorporating one or more near-field spatial filters 102a-c and/or far-field spatial filters 110n-c.

FIG. 1B illustrates an example of a lens to filter light reflected from (or otherwise returned/directed from) near-field and far-field objects. More specifically, FIG. 1B depicts eyeglass 190 comprising lens 100 in a pair of glasses 192 configured to filter light reflected from (or otherwise returned/directed from) near-field and far-field objects. Lens 100 may comprise a number of near-field spatial filters 102a-n and far-field spatial filters 110a-n arranged to provide eyeglass 190 with multiple focal ranges (e.g., a bi-focal eyeglass). Near-field spatial filters 102a-n may be disposed at a bottom portion of lens 100 in a greater number than a number of far-field spatial filters 110a-n disposed on the bottom portion of lens 100. A number of near-field spatial filters 102a-n disposed at a top portion of lens 100 may be less than a number of far-field spatial filters 110a-n disposed on the top portion of lens 100.

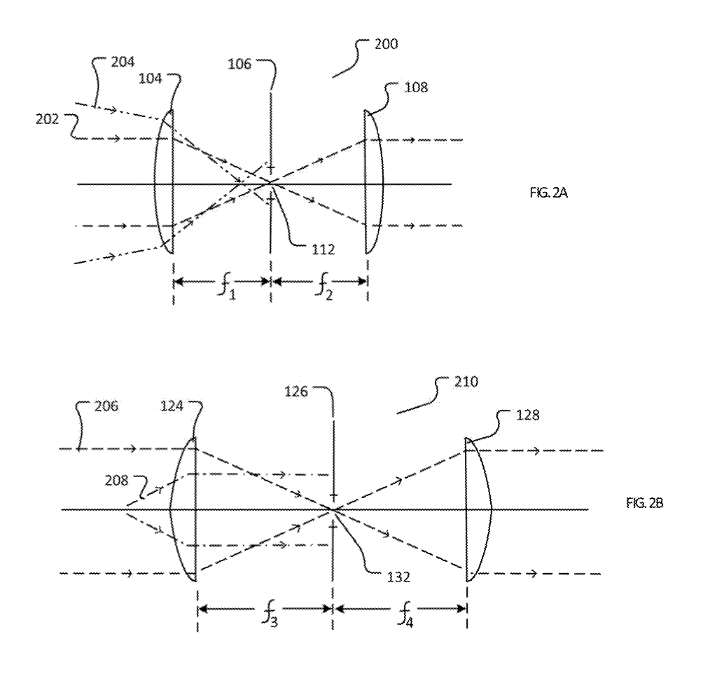

FIG. 2A is a diagram of example focal distances of a filter to filter light reflected from (or otherwise returned/directed from) near-field and far-field objects in an example lens, and more specifically, FIG. 2A is a diagram 200 illustrating an example of first focal length f1 of first near-field lens 104 and second focal length f2 of second near-field lens 108. First focal length f1 may be a back focal length of first near-field lens 104, and second focal length f2 may be a front focal length of second near-field lens 108. The term “parallel light” may refer to light that is parallel to an entrance of lens 100. Parallel or near parallel light of light 202 may be reflected from (or otherwise returned/directed from) a near-field object and may be refracted (or otherwise directed) by first near-field lens 104 through pinhole 112 of first light filter 106. Divergent light of light 204 may be reflected from (or otherwise returned/directed from) a far-field object and may be refracted away from (or otherwise directed away from) pinhole 112 such that most of a divergent light portion of light 204 will be blocked by first light filter 106 and not allowed to pass to second near-field lens 108 via pinhole 112. Some portion of light 204 may pass through pinhole 112. In such a case, the power of light 204 may be scattered by second near-field lens 108, and thereby a substantial portion of the power of divergent light of light 204 may not enter into an optical image system (such as optical image system 150) associated with lens 100.

FIG. 2B is a diagram of example focal distances of a filter to filter light reflected from (or otherwise returned/directed from) near-field and far-field objects in an example lens, and more specifically, FIG. 2B is a diagram 210 illustrating an example of a third focal length 13 of first far-field lens 124 and fourth focal length f4 of second far-field lens 128. Third focal length 13 may be a back focal length of first far-field lens 124 and fourth focal length f4 may be a front focal length of second far-field lens 128. Parallel or near parallel light of light 206 may be reflected from (or otherwise returned/directed from) a far-field object and may be incident on first far-field lens 124. Light 206 may be refracted (or otherwise directed) by first far-field lens 124 through pinhole 132 of second light filter 126. Divergent light of light 208 may be reflected from (or otherwise returned/directed from) a near-field object and may be incident on first far-field lens 124. Light 208 may be refracted away from (or otherwise directed away from) pinhole 132 such that most of light 208 will be blocked and not allowed to pass to second far-field lens 128 via pinhole 132. Some portion of light 208 may pass through pinhole 132. In such a case, the power of light 208 may be scattered by second far-field lens 128, and thereby a substantial portion of the power of divergent light of light 208 may not enter into an optical image system (such as optical image system 150) associated with lens 100.

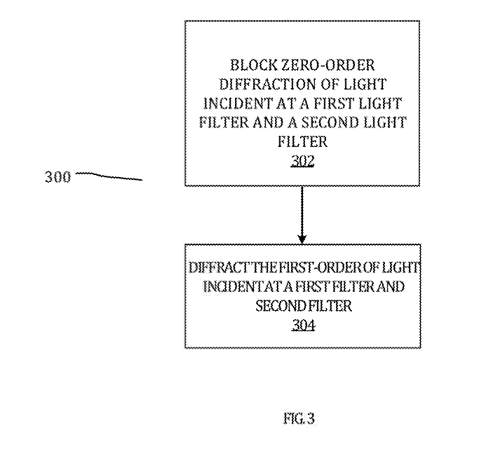

FIG. 3 illustrates a flowchart of an example process to filter light reflected from (or otherwise returned/directed from) near-field and far-field objects in an example lens. More specifically, FIG. 3 illustrates a flowchart of an example process 300 arranged to filter light reflected from (or otherwise returned/directed from) near-field and far-field objects in an example lens, arranged in accordance with at least some embodiments described herein. A light field lens (such as the previously described lens 100 and its associated components) in one embodiment may include a first spatial filter, the first spatial filter including a first lens having a first focal length, a second lens having a second focal length, and a first light filter (which in one embodiment may comprise a first optical processing filter), the first optical processing filter being disposed between the first lens and the second lens such that the first optical processing filter is substantially at a point where the focus of the first lens and the focus of the second lens substantially coincide based at least in part on the first focal length and the second focal length. The light field lens may further include a second spatial filter optically coupled to the first spatial filter, the second spatial filter including a third lens having a third focal length, a fourth lens having a fourth focal length, and a second light filter (which in one embodiment may comprise a second optical processing filter), the second optical processing filter being disposed between the third lens and the fourth lens such that the second optical processing filter is substantially at a point where the focus of the third lens and the focus of the fourth lens substantially coincide based at least in part on third focal length and the fourth focal length. As depicted, process 300 may start at operation 302 (“Block Zero-Order Diffraction Of Light Incident At A First Light Filter And Second Light Filter”), where zero-order diffraction of light incident at the first light filter and the second light filter may be blocked by the first and second light filters, respectively. Process 300 may move to operation 304 (“Diffract The First-Order Of Light Incident At A First Filter And Second Filter”), where the first-order diffraction (or other passage) of light incident at the first optical processing filter and the second optical processing filter may be allowed (e.g., not blocked).

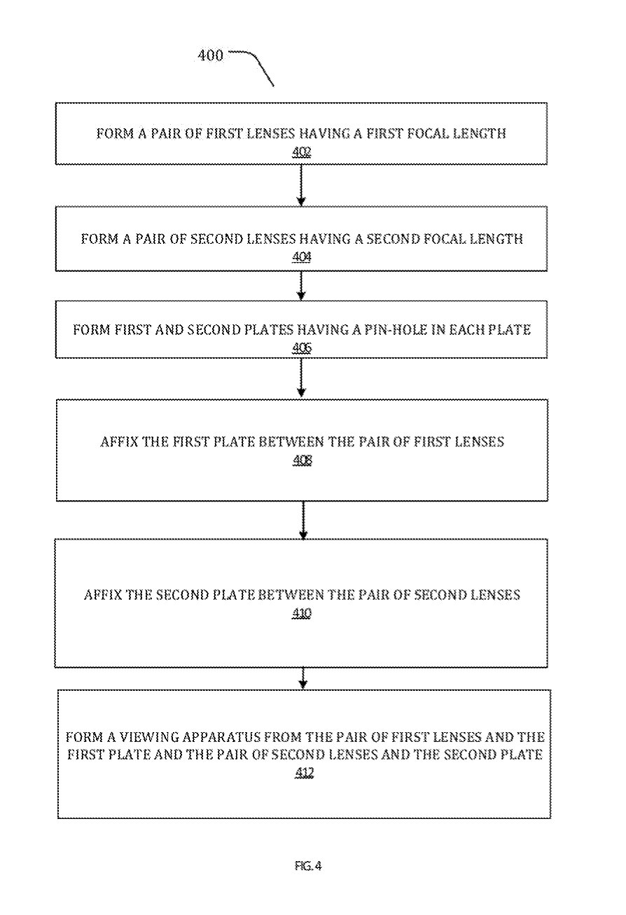

FIG. 4 illustrates a flowchart of an example process to make a lens to filter light reflected from (or otherwise returned/directed from) near-field and far-field objects in an example lens. More specifically, FIG. 4 illustrates a flowchart of an example process 400 arranged to make a lens to filter light reflected from (or otherwise returned/directed from) near-field and far-field objects in an example lens, arranged in accordance with at least some embodiments described herein. The process 400 (as well as any other process, method, etc. described elsewhere in this disclosure) may include one or more operations or actions, as illustrated by one or more of blocks. Although illustrated as discrete blocks, various blocks may be divided into additional blocks, combined into fewer blocks, or eliminated, depending on the particular implementation. Blocks may be supplemented with additional blocks representing other operations or actions.

Process 400 may begin at block 402 (“Form A Pair Of First Lenses Having A First Focal Length”) where, a pair of first lenses having a first focal length may be formed. Process 400 may move to block 404 (“Form A Pair Of Second Lenses Having A Second Focal Length”) where a pair of second lenses having a second focal length may be formed. Process 400 may move to block 406 (“Form First And Second Plates Having A Pin-Hole In Each Plate”) where two plates having a pin-hole in each plate may be formed. Process 400 may move to block 408 (“Affix The First Plate Between The Pair Of First Lenses”) where the first plate may be affixed between the pair of first lenses such that the pin-hole in the plate may be substantially at a point where a focus (corresponding to the first focal length) of the pair of first lenses substantially coincide. Process 400 moves to block 410 (“Affix The Second Plate Between The Pair Of Second Lenses”) where the second plate may be affixed between the pair of second lenses such that the pin-hole in the plate may be substantially at a point where a focus (corresponding to the second focal length) of the pair of second lenses substantially coincide. Process 400 may move to block 412 (“Form A Viewing Apparatus From The Pair Of First Lenses And The First Plate And The Pair Of Second Lenses And The Second Plate”) where a viewer apparatus may be formed from the pair of first lenses including the first plate and the pair of second lenses including the second plate, wherein the viewer apparatus optically couples the pair of first lenses including the first plate and the pair of second lenses including the second plate.

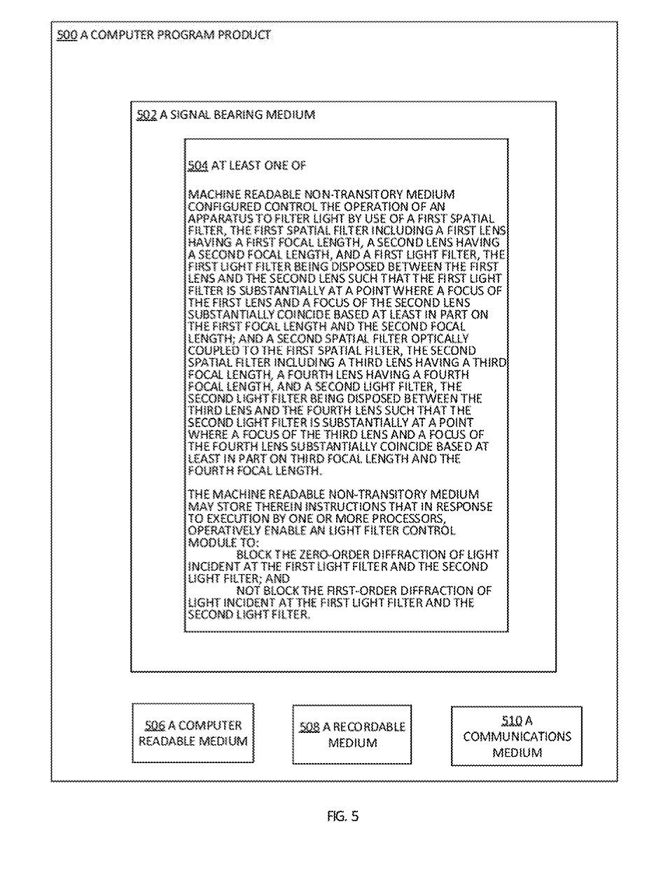

FIG. 5 illustrates an example computer program product to control or otherwise operate in conjunction with an example lens to filter light reflected from (or otherwise returned/directed from) near-field and far-field objects. More specifically, FIG. 5 illustrates an example computer program product 500 configured to control or otherwise operate in conjunction with an example lens to filter light reflected from (or otherwise returned/directed from) near-field and far-field objects, arranged in accordance with at least some embodiments described herein. As depicted, computer program product 500 may include a machine-readable non-transitory medium configured to control the operation of an apparatus (such as lens 100 described above) to filter light, by use of a first spatial filter, the first spatial filter including a first lens having a first focal length, a second lens having a second focal length, and a first light filter (which in one embodiment may comprise a first optical processing filter), the first light filter being disposed between the first lens and the second lens such that the first light filter is substantially at a point where a focus of the first lens and a focus of the second lens substantially coincide based at least in part on the first focal length and the second focal length, and a second spatial filter optically coupled to the first spatial filter, the second spatial filter including a third lens having a third focal length, a fourth lens having a fourth focal length, and a second light filter (which in one embodiment may comprise a second optical processing filter), the second light filter being disposed between the third lens and the fourth lens such that the second light filter is substantially at a point where a focus of the third lens and a focus of the fourth lens substantially coincide based at least in part on third focal length and the fourth focal length. The machine readable non-transitory medium may store therein instructions that, in response to execution by one or more processors, operatively enable a light filter control module (such as the optical processing filter control module 130 described above) to block the zero-order diffraction of light incident at the first light filter and the second light filter and not block the first-order diffraction (or other passage) of light incident at the first light filter and the second light filter.

Computer program product 500 may include a signal bearing medium 502. Signal bearing medium 502 may include one or more machine-readable instructions 504, which, in response to execution by one or more processors, may operatively enable a computing device to provide the features and/or perform the operations (including controlling the operations) described herein. In various examples, the devices discussed herein may use some or all of the machine-readable instructions.

In one example, the machine-readable instructions 504 may include instructions to block the zero-order diffraction of light incident at the first light filter and the second light filter. In some examples, the machine-readable instructions 504 may include instructions to allow the first-order diffraction of light incident at the first light filter and the second light filter.

In one example, signal bearing medium 502 may encompass a computer-readable medium 506, such as, but not limited to, a hard disk drive, a Compact Disc (CD), a Digital Versatile Disk (DVD), a digital tape, memory, etc. In some implementations, signal bearing medium 502 may encompass a recordable medium 508, such as, but not limited to, memory, read/write (R/W) CDs, R/W DVDs, etc. In some implementations, signal bearing medium 502 may encompass a communications medium 510, such as, but not limited to, a digital and/or an analog communication medium (e.g., a fiber optic cable, a waveguide, a wired communication link, a wireless communication link, etc.). In some examples, signal bearing medium 502 may encompass a machine readable non-transitory medium.

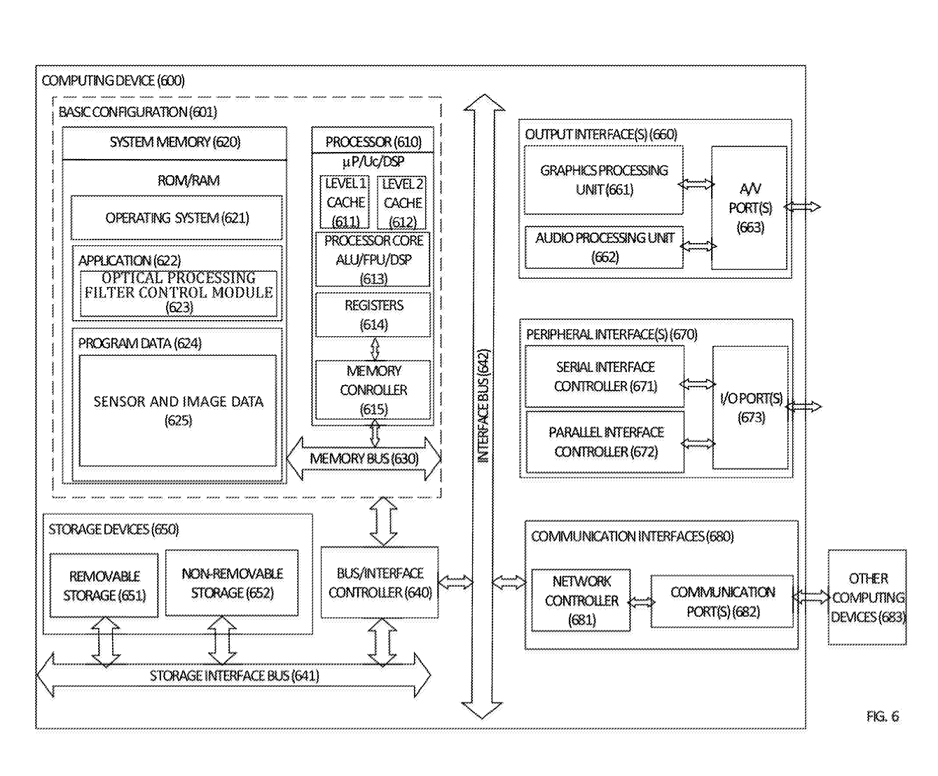

In general, the method described with respect to FIGS. 1-5, and elsewhere herein may be implemented in some instances in any suitable server and/or computing system. Example systems may be described with respect to FIG. 6 and elsewhere herein. In general in some embodiments, the computer system may be configured to perform or cause to be performed operations to block zero-order diffraction of light incident at a first light filter and a second light filter and allow first-order diffraction of light incident at the first optical processing filter and the second optical processing filter.

FIG. 6 illustrates a block diagram of an example computing device, arranged in accordance with at least some embodiments described herein. More specifically, FIG. 6 is a block diagram illustrating an example of a computing device 600, arranged in accordance with at least some embodiments of the present disclosure. In various examples, computing device 600 may be configured to facilitate or otherwise control or operate in conjunction with blocking zero-order diffraction of light incident at a first light filter and a second light filter and allowing first-order diffraction of light incident at the first light filter and the second light filter, as discussed herein. In one example of a basic configuration 601, computing device 600 may include one or more processors 610 and a system memory 620. A memory bus 630 can be used for communicating between one or more processors 610 and system memory 620.

Depending on the desired configuration, one or more processors 610 may be of any type including but not limited to a microprocessor (μP), a microcontroller (μC), a digital signal processor (DSP), or any combination thereof. One or more processors 610 may include one or more levels of caching, such as a level one cache 611 and a level two cache 612, a processor core 613, and registers 614. Processor core 613 can include an arithmetic logic unit (ALU), a floating point unit (FPU), a digital signal processing core (DSP core), or any combination thereof. A memory controller 615 can also be used with one or more processors 610, or in some implementations memory controller 615 can be an internal part of processor 610.

Depending on the desired configuration, system memory 620 may be of any type including but not limited to volatile memory (such as RAM), non-volatile memory (such as ROM, flash memory, etc.) or any combination thereof. System memory 620 may include an operating system 621, one or more applications 622, and program data 624. One or more applications 622 may include an optical processing filter control module application 623 that may be arranged to perform the actions and/or operations as described herein, including the actions and/or operations to facilitate blocking zero-order diffraction of light incident at a first light filter and a second light filter and allowing first-order diffraction of light incident at the first light filter and the second light filter, as described herein. Program data 624 may include, among other data, sensor data and/or image data 625 or the like for use with optical processing filter control module application 623, as described herein. In some example embodiments, one or more applications 622 may be arranged to operate with program data 624 on operating system 621. This described basic configuration 601 is illustrated in FIG. 6 by those components within a dashed line.

The computing device 600 may have additional features or functionality, and additional interfaces to facilitate communications between basic configuration 601 and any required devices and interfaces. For example, a bus/interface controller 640 may be used to facilitate communications between basic configuration 601 and one or more data storage devices 650 via a storage interface bus 641. One or more data storage devices 650 may be removable storage devices 651, non-removable storage devices 652, or a combination thereof. Examples of removable storage and non-removable storage devices include magnetic disk devices such as flexible disk drives and hard-disk drives (HDDs), optical disk drives such as compact disk (CD) drives or digital versatile disk (DVD) drives, solid state drives (SSDs), and tape drives to name a few. Example computer storage media may include volatile and nonvolatile, removable and non-removable media implemented in any method or technology for storage of information, such as computer readable instructions, data structures, program modules, or other data. System memory 620, removable storage 651 and non-removable storage 652 are all examples of computer storage media. The computer storage media includes, but is not limited to, RAM, ROM, EEPROM, flash memory or other memory technology, CD-ROM, digital versatile disks (DVDs) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

The computing device 600 may also include an interface bus 642 for facilitating communication from various interface devices (e.g., output interfaces, peripheral interfaces, and communication interfaces) to basic configuration 601 via bus/interface controller 640. Example output interfaces 660 may include a graphics processing unit 661 and an audio processing unit 662, which may be configured to communicate to various external devices such as a display or speakers via one or more A/V ports 663. Example peripheral interfaces 670 may include a serial interface controller 671 or a parallel interface controller 672, which may be configured to communicate with external devices such as input devices (e.g., keyboard, mouse, pen, voice input device, touch input device, etc.) or other peripheral devices (e.g., printer, scanner, etc.) via one or more I/O ports 673. An example communication interface 680 includes a network controller 681, which may be arranged to facilitate communications with one or more other computing devices 683 over a network communication via one or more communication ports 682. A communication connection is one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), infrared (IR) and other wireless media. The term computer readable media as used herein may include both storage media and communication media.

The computing device 600 may be implemented as a portion of a small-form factor portable (or mobile) electronic device such as a cell phone, a mobile phone, a tablet device, a laptop computer, a personal data assistant (PDA), a personal media player device, a wireless web-watch device, a personal headset device, an application specific device, or a hybrid device that includes any of the above functions. Computing device 600 may also be implemented as a personal computer including both laptop computer and non-laptop computer configurations. In addition, computing device 600 may be implemented as part of a wireless base station or other wireless system or device.

Some portions of the foregoing detailed description are presented in terms of algorithms or symbolic representations of operations on data bits or binary digital signals stored within a computing system memory, such as a computer memory. These algorithmic descriptions or representations are examples of techniques used by those of ordinary skill in the data processing arts to convey the substance of their work to others skilled in the art. An algorithm is here, and generally, is considered to be a self-consistent sequence of operations or similar processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals or the like. It should be understood, however, that all of these and similar terms are to be associated with appropriate physical quantities and are merely convenient labels. Unless specifically stated otherwise, as apparent from the following discussion, it is appreciated that throughout this specification discussions utilizing terms such as “processing,” “computing,” “calculating,” “determining” or the like refer to actions or processes of a computing device, that manipulates or transforms data represented as physical electronic or magnetic quantities within memories, registers, or other information storage devices, transmission devices, or display devices of the computing device.

The claimed subject matter is not limited in scope to the particular implementations described herein. For example, some implementations may be in hardware, such as employed to operate on a device or combination of devices, for example, whereas other implementations may be in software and/or firmware. Likewise, although claimed subject matter is not limited in scope in this respect, some implementations may include one or more articles, such as a signal bearing medium, a storage medium and/or storage media. This storage media, such as CD-ROMs, computer disks, flash memory, or the like, for example, may have instructions stored thereon, that, when executed by a computing device, such as a computing system, computing platform, or other system, for example, may result in execution of a processor in accordance with the claimed subject matter, such as one of the implementations previously described, for example. As one possibility, a computing device may include one or more processing units or processors, one or more input/output devices, such as a display, a keyboard and/or a mouse, and one or more memories, such as static random access memory, dynamic random access memory, flash memory, and/or a hard drive.

The use of hardware or software is generally (but not always, in that in certain contexts the choice between hardware and software can become significant) a design choice representing cost vs. efficiency tradeoffs. There are various vehicles by which processes and/or systems and/or other technologies described herein can be affected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware are possible in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a flexible disk, a hard disk drive (HDD), a Compact Disc (CD), a Digital Versatile Disk (DVD), a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to subject matter containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Reference in the specification to “an example,” “one example,” “some examples,” or “other examples” may mean that a particular feature, structure, or characteristic described in connection with one or more examples may be included in at least some examples, but not necessarily in all examples. The various appearances of “an example,” “one example,” or “some examples” in the preceding description are not necessarily all referring to the same example.

While certain exemplary techniques have been described and shown herein using various methods and systems, various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular examples disclosed, but that such claimed subject matter also may include all implementations falling within the scope of the appended claims, and equivalents thereof.

1. A lens apparatus, comprising:
a plurality of near-field spatial filters, at least some of the near-field spatial filters including a first near-field lens that includes a first focal length, a second near-field lens that includes a second focal length, and a first plate that includes a pin-hole, the first plate being disposed between the first near-field lens and the second near-field lens such that the pin-hole in the first plate is located substantially at a point where a focus of the first near-field lens and a focus of the second near-field lens substantially coincide based at least in part on the first focal length and the second focal length; and
a plurality of far-field spatial filters optically coupled to the plurality of near-field spatial filters, at least some of the far-field spatial filters including a first far-field lens that includes a third focal length, a second far-field lens that includes a fourth focal length, and a second plate that includes a pin-hole, the second plate being disposed between the first far-field lens and the second far-field lens such that the pin-hole in the second plate is located substantially at a point where a focus of the first far-field lens and a focus of the second far-field lens substantially coincide based at least in part on the third focal length and the fourth focal length, and wherein the third focal length and the fourth focal length are different than either of the first focal length and the second focal length.

2. The lens apparatus of claim 1, wherein the first focal length and the second focal length are a same length and wherein the third focal length and the fourth focal length are a same length.

3. The lens apparatus of claim 1, wherein the first focal length and the second focal length are shorter than the third focal length and the fourth focal length.

4. The lens apparatus of claim 3, wherein the first focal length is arranged such that an object less than approximately 6.0 meters away from the far-field lens is substantially in focus at the pin-hole in the first plate.

5. The lens apparatus of claim 3, wherein the third focal length is arranged such that an object greater than approximately 6 meters away from the far-field lens is substantially in focus at the pin-hole in the second plate.

6. The lens apparatus of claim 1, wherein the plurality of near-field spatial filters and the plurality of far-field spatial filters are arranged in an array of alternating near-field spatial filters and far-field spatial filters.

7. The lens apparatus of claim 6, wherein a number of near-field filters and far-field filters is approximately equal.

8. The lens apparatus of claim 1, wherein the plurality of near-field spatial filters and far-field spatial filters are included in a contact lens.

9. The lens apparatus of claim 1, wherein the plurality of near-field spatial filters and far-field spatial filters are included in an intraocular lens.

10. The lens apparatus of claim 3, wherein the plurality of near-field spatial filters and far-field spatial filters are included in an eye glass lens, and wherein a number of individual ones of the plurality of near-field filters at a bottom half of the eye glass lens is greater than a number of individual ones of the plurality of far-field filters at the bottom half of the eye glass lens, and wherein a number of individual ones of the plurality of near-field filters at a top half of the eye glass lens is less than a number of individual ones of the plurality of far-field filters at the top half of the eye glass lens.

11. The lens apparatus of claim 1, further comprising:
an image inversion lens configured to invert images viewed through the plurality of near-field spatial filters and the plurality of far-field spatial filters.

12. An apparatus, comprising:
a first spatial filter, the first spatial filter including a first lens that includes a first focal length, a second lens that includes a second focal length, and a first plate that includes a pin-hole, the first plate being disposed between the first lens and the second lens such that the pin-hole in the first plate is located substantially at a point where a focus of the first lens and a focus of the second lens substantially coincide based at least in part on the first focal length and the second focal length; and
a second spatial filter optically coupled to the first spatial filter, the second spatial filter including a third lens that includes a third focal length, a fourth lens that includes a fourth focal length, and a second plate that includes a pin-hole, the second plate being disposed between the third lens and the fourth lens such that the pin-hole in the second plate is located substantially at a point where a focus of the third lens and a focus of the fourth lens substantially coincide based at least in part on the third focal length and the fourth focal length.

13. The apparatus of claim 12, wherein the first focal length, the second focal length, the third focal length, and the fourth focal length are substantially a same length.

14. The apparatus of claim 12, wherein the first focal length and the second focal length area different length than the third focal length and the fourth focal length.

15. The apparatus of claim 12, wherein the first focal length and the second focal length are shorter than the third focal length and the fourth focal length.

16. The apparatus of claim 12, further comprising:
a telescope, wherein the first spatial filter and the second spatial filter are incorporated into a viewer apparatus of the telescope.

17. The apparatus of claim 12, further comprising:
a microscope, wherein the first spatial filter and the second spatial filter are incorporated into a viewer apparatus of the microscope.

18. A system, comprising:
a first spatial filter, the first spatial filter including a first lens that includes a first focal length, a second lens that includes a second focal length, and a first optical processing filter, the first optical processing filter being disposed between the first lens and the second lens such that the optical processing filter is located substantially at a point where a focus of the first lens and a focus of the second lens substantially coincide based at least in part on the first focal length and the second focal length;
a second spatial filter optically coupled to the first spatial filter, the second spatial filter including a third lens that includes a third focal length, a fourth lens that includes a fourth focal length, and a second optical processing filter, the second optical processing filter being disposed between the third lens and the fourth lens such that the second optical processing filter is located substantially at a point where a focus of the third lens and a focus of the fourth lens substantially coincide based at least in part on the third focal length and the fourth focal length; and
an optical processing filter control module operatively coupled to the first and second optical processing filters, the optical processing filter control module operatively enabled to:
block a zero-order diffraction of light incident at the first optical processing filter and the second optical processing filter; and
enable first-order diffraction of light incident at the first optical processing filter and the second optical processing filter.

19. The system of claim 18, wherein the first focal length, the second focal length, the third focal length, and the fourth focal length are substantially a same length.

20. The system of claim 18, wherein the first focal length and the second focal length are a different length than the third focal length and the fourth focal length.

21. The system of claim 20, wherein the first focal length and the second focal length are shorter than the third focal length and the fourth focal length.

22. A method to filter light by use of a first spatial filter and a second spatial filter,
the first spatial filter including a first lens that includes a first focal length, a second lens that includes a second focal length, and a first optical processing filter, the first optical processing filter being disposed between the first lens and the second lens such that the first optical processing filter is located substantially at a point where a focus of the first lens and a focus of the second lens substantially coincide based at least in part on the first focal length and the second focal length; and
the second spatial filter optically coupled to the first spatial filter, the second spatial filter including a third lens that includes a third focal length, a fourth lens that includes a fourth focal length, and a second optical processing filter, the second optical processing filter being disposed between the third lens and the fourth lens such that the second optical processing filter is located substantially at a point where a focus of the third lens and a focus of the fourth lens substantially coincide based at least in part on the third focal length and the fourth focal length, the method comprising:
blocking a zero-order diffraction of light incident at the first optical processing filter and the second optical processing filter; and
enabling a first-order diffraction of light incident at the first optical processing filter and the second optical processing filter.

23. The method of claim 22, further comprising:
directing, by the first spatial filter, the light, which comprises a first parallel light and a first divergent light reflected from a near-field object, from the first lens through the first optical processing filter to the second lens;
wherein directing further comprises:
directing, by the second lens, the first parallel light that passes through the first optical processing filter to an optical image system;
blocking, by the first optical processing filter, a first portion of the first divergent light from the first lens;
scattering, by the second lens, a second portion of the first divergent light that passes through the first optical processing filter; and
directing, by the second spatial filter, the light, which comprises a second parallel light and a second divergent light reflected from a far-field object, from the third lens through the second optical processing filter to the fourth lens;
wherein directing further comprises:
directing, by the third lens, the second parallel light to the optical image system;
blocking, by the second optical processing filter, a first portion of the second divergent light from the third lens; and
scattering, by the fourth lens, a second portion of the second divergent light that is not blocked.

24. (canceled)

25. A process to manufacture a micro lens array, the process comprising:
forming a pair of first lenses that include a first focal length;
forming a pair of second lenses that include a second focal length;
forming first and second plates that include a pin-hole in each plate;
affixing the first plate between the pair of first lenses such that the pin-hole in the first plate is substantially at a point where a focus of the pair of first lenses substantially coincide; and
affixing the second plate between the pair of second lenses such that the pin-hole in the second plate is substantially at a point where a focus of the pair of second lenses substantially coincide.

26. The process of claim 25, further comprising forming a viewer apparatus from the pair of first lenses including the first plate and the pair of second lenses including the second plate, wherein the viewer apparatus is configured to optically couple the pair of first lenses including the first plate and the pair of second lenses including the second plate.

27. The process of claim 26, wherein the viewer apparatus comprises a telescope, a microscope, a window, a contact lens, a lens, glasses, goggles, or a combination thereof.

28. The process of claim 25, wherein either or both of the pair of first lenses and the pair of second lenses comprises a gradient-index (GRIN) lens, a Fresnel lens, a Fresnel zone plate, or a combination thereof.
